(19) United States
(12) Patent Application Publication
(10) Pub. No.: US 2022/0354425 A1
(43) Pub. Date: Nov. 10, 2022

(54) DIELECTRIC TRANSMISSION PROBES AND METHODS OF USING THE SAME

(71) Applicant: Trustees of Dartmouth College, Hanover, NH (US)

(72) Inventors: Paul Meaney, Hanover, NH (US); Timothy Raynolds, Lebanon, NH (US); Robin Augustine, Uppsala (SE)

(73) Assignee: Trustees of Dartmouth College, Hanover, NH (US)

(21) Appl. No.: 17/766,121

(22) PCT Filed: Oct. 2, 2020
(86) PCT No.: PCT/US2020/054065

§ 371 Date: Apr. 1, 2022

Related U.S. Application Data
(60) Provisional application No. 62/909,620, filed on Oct. 2, 2019.

(51) Int. Cl. A61B 5/00; A61B 18/18

(52) U.S. Cl. CPC A61B 5/4869; A61B 18/18

(57) ABSTRACT

Embodiments of the present disclosure pertain to transmission dielectric probes with at least one channel that extends across the probe. The channel includes a first opening on a first side of the transmission dielectric probe, and a second opening on a second side of the transmission dielectric probe. The first opening and the second opening are on opposite ends of the transmission dielectric probe, and the second opening is associated with an outer surface of the transmission dielectric probe. Additionally, the first opening and the second opening have different diameters, different geometries, or combinations thereof. Further embodiments pertain to methods of operating the transmission dielectric probes by placing the outer surface of the transmission dielectric probe on a surface of an object, transmitting a signal from a first channel through the surface and into the object, and receiving the transmitted signal back through a second channel.

### CROSS-REFERENCE TO RELATED APPLICATIONS

This application claims priority to U.S. Provisional Patent Application No. 62/909,620, filed on Oct. 2, 2019. The entirety of the aforementioned application is incorporated herein by reference.

### STATEMENT REGARDING FEDERALLY SPONSORED RESEARCH

This invention was made with government support under R01 CA191227 and R01 CA240760 awarded by the National Institutes of Health. The government has certain rights in the invention.

### BACKGROUND

Current transmission dielectric probes suffer from numerous limitations, such as poor penetration depth, sensitivity to cable movement, large sizes, and high prices. Numerous embodiments of the present disclosure address the aforementioned limitations.

### SUMMARY

In some embodiments, the present disclosure pertains to transmission dielectric probes with at least one channel that extends across the transmission dielectric probe. The at least one channel includes: (1) a first opening on a first side of the transmission dielectric probe, and (2) a second opening on a second side of the transmission dielectric probe. The first opening and the second opening are on opposite ends of the transmission dielectric probe, and the second opening is associated with an outer surface of the transmission dielectric probe. Additionally, the first opening and the second opening have different diameters, different geometries, or combinations thereof.

In some embodiments, the diameter of the first opening of the transmission dielectric probes of the present disclosure is smaller than the diameter of the second opening. In some embodiments, the diameter of the channel becomes gradually wider as it extends from the first opening to the second opening. In some embodiments, the first opening is in the form of a circle, and the second opening is in the form of an ellipse.

In some embodiments, the at least one channel is enclosed within the transmission dielectric probes of the present disclosure. In some embodiments, the at least one channel includes an inner conductor and an outer conductor that is coaxial with the inner conductor. In some embodiments, the inner conductor is not positioned at the center of the channel.

In some embodiments, the transmission dielectric probes of the present disclosure include at least two adjacent channels. In some embodiments, the at least two adjacent channels have the same channel shape.

In some embodiments, the transmission dielectric probes of the present disclosure also include one or more analyzers. In some embodiments, the one or more analyzers are associated with the transmission dielectric probes of the present disclosure through one or more cables.

Additional embodiments of the present disclosure pertain to methods of operating the transmission dielectric probes of the present disclosure by (1) placing the outer surface of the transmission dielectric probe on a surface of the object; (2) transmitting a signal from a first channel through the surface and into the object; and (3) receiving the transmitted signal back through a second channel. The methods of the present disclosure may also include a step of utilizing an analyzer to display the transmitted signals for evaluation. Additionally, the methods of the present disclosure may include a step of utilizing the transmitted signal to evaluate a property of the object.

In some embodiments, the evaluation includes a calculation of a property of the object (e.g., dielectric properties of the object). In some embodiments, the evaluation includes the characterization of the object directly from the transmitted signals. In some embodiments, a machine learning algorithm may be utilized to evaluate a property of the object.

In some embodiments, the object is a tissue of a subject. As such, in some embodiments, the methods of the present disclosure can be utilized to evaluate a property of the tissue, make a treatment decision based on the evaluated property, and then treat a disease or condition in the subject.

### DRAWINGS

FIG. 1A provides a depiction of a transmission dielectric probe in accordance with one embodiment of the present disclosure.

FIG. 1B provides a method of utilizing a transmission dielectric probe to obtain transmission signals from an object in accordance with one embodiment of the present disclosure.

FIG. 1C provides a method of utilizing a transmission dielectric probe to obtain transmission signals from a tissue in accordance with one embodiment of the present disclosure.

FIG. 2 provides another depiction of a transmission dielectric probe in accordance with one embodiment of the present disclosure.

FIG. 3 provides another depiction of a transmission dielectric probe in accordance with one embodiment of the present disclosure.

FIGS. 4A and 4B show repeatability measurements (magnitude and phase) for transmission dielectric probes.

FIGS. 5A and 5B show results regarding the use of transmission dielectric probes for measurements in different liquids.

FIGS. 6A and 6B show results regarding the use of transmission dielectric probes for measurements at different penetration depths.

FIGS. 7A and 7B show results regarding the use of transmission dielectric probes for making various body tissue measurements.

FIGS. 8A and 8B show data pertaining to the use of transmission dielectric probes to evaluate different sites of a body.

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that comprise more than one unit unless specifically stated otherwise.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

Numerous commercial reflection-based transmission dielectric probes exist. However, such probes are relegated exclusively to bench-type measurements for a number of critical reasons, such as primarily poor penetration depth and the fact that even minute perturbations of the connecting coaxial feedlines can disrupt the measurements to a degree where they are rendered non-usable.

At the same time, there have been major challenges for translating microwave sensing technologies into the clinic. For instance, while full-fledged microwave imaging systems are being developed, such systems are relatively large, bulky and expensive.

Moreover, commercial, reflection-based coaxial probes have been available for several decades. However, such probes are vulnerable to obtaining artifacts from even the slightest movement of connecting cables.

Additionally, commercial probes have suffered from poor penetration depth, which is a critical factor for interrogating any relevant tissue. In particular, a tissue is generally covered with a thin layer of skin followed by a subcutaneous layer of fat. For a probe to be effective, it must penetrate at least through these layers to provide information regarding the underlying tissue.

Such limitations have had clinical repercussions. For instance, full-fledged breast cancer screening programs utilize handheld ultrasound devices in remote settings. However, such ultrasound devices are not approved for breast imaging. Moreover, such ultrasound devices have trouble distinguishing between normal tissue and breast cancer.

Accordingly, a need exists for the development of probes that have improved penetration depths. A need also exists for the development of probes that are less sensitive to the movement of connecting cables. In addition, a need exists for the development of probes that are more compact and affordable. A need also exists for the development of probes that can distinguish between different fluids and tissues. Embodiments of the present disclosure address the aforementioned needs.

In some embodiments, the present disclosure pertains to transmission dielectric probes. In some embodiments, the transmission dielectric probes of the present disclosure include at least one channel that extends across the transmission dielectric probe. In some embodiments, the at least one channel includes a first opening on a first side of the transmission dielectric probe, and a second opening on a second side of the transmission dielectric probe. In some embodiments, the first opening and the second opening are on opposite ends of the transmission dielectric probe, and the second opening is associated with an outer surface of the transmission dielectric probe. In some embodiments, the first opening and the second opening have different diameters, different geometries, or combinations thereof.

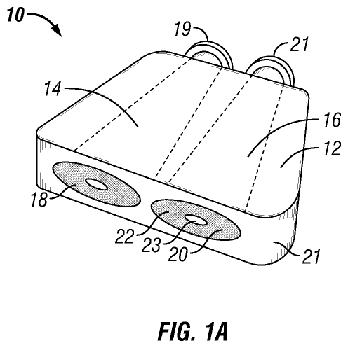

In some embodiments, the transmission dielectric probes of the present disclosure include at least two channels. In some embodiments, the at least two channels are adjacent to one another. In more specific embodiments illustrated in FIG. 1A, the transmission dielectric probes of the present disclosure are in the form of transmission dielectric probe 10. In this embodiment, transmission dielectric probe 10 includes a body 12 that houses a first channel 14 and an adjacent second channel 16. In the embodiment shown in FIG. 1A, first channel 14 contains an opening 18 and an opening 19, where opening 19 is narrower than opening 18. Similarly, second channel 16 contains an opening 20 and an opening 21, where opening 21 is narrower than opening 20. Openings 18 and 20 are on a straight and flat outer surface of transmission dielectric probe 10.

In the embodiment shown in FIG. 1A, each of the channels 14 and 16 has an inner conductor 23 and an outer conductor 22. In this embodiment, the inner and outer conductors of the first and second channels transition from circular shapes to elliptical shapes.

Additional embodiments of the present disclosure pertain to methods of operating the transmission dielectric probes of the present disclosure. In some embodiments illustrated in FIG. 1B, the methods of the present disclosure include placing the outer surface of a transmission dielectric probe of the present disclosure on a surface of an object (step 30), transmitting a signal from a first channel of the transmission dielectric probe through the surface and into the object (step 32), and then receiving the transmitted signal back through a second channel of the transmission dielectric probe (step 34). In some embodiments, the methods of the present disclosure also include a step of utilizing an analyzer to display the transmitted signal (step 36). In some embodiments, the methods of the present disclosure also include a step of utilizing the transmitted signal to evaluate a property of the object (step 38).

For instance, in some embodiments illustrated in FIG. 1A, openings 18 and 20 are placed on a surface of an object. Additionally, an analyzer may be connected to openings 19 and 21 and used to display transmitted signals on a screen for evaluation.

In some embodiments, the objects to be evaluated include tissues. As such, in more specific embodiments illustrated in FIG. 1C, the methods of the present disclosure can include one or more of the following steps: placing the outer surface of a transmission dielectric probe of the present disclosure on a tissue's surface (step 40), transmitting a signal from a first channel of the transmission dielectric probe through the surface and into the tissue (step 42), receiving the transmitted signal back through a second channel (step 44), displaying the transmitted signals (step 46), utilizing the transmitted signal to evaluate a property of the tissue (step 48), making a treatment decision based on the evaluated property of the tissue (step 50), and treating a disease or a condition based on the evaluated property of the tissue (step 52).

Additional embodiments of the present disclosure pertain to methods of fabricating the transmission dielectric probes of the present disclosure. As set forth in more detail herein, the transmission dielectric probes of the present disclosure can have numerous structures, applications and methods of fabrication.

Channels

The channels of the transmission dielectric probes of the present disclosure generally extend across the transmission dielectric probes. The transmission dielectric probes of the present disclosure can include numerous types of channels. For instance, in some embodiments, the channels of the present disclosure are enclosed within the transmission dielectric probes of the present disclosure. In some embodiments, the channels are in the form of apertures. In some embodiments, the channels have an elliptical shape. In some embodiments, the channels have a cone-like shape. In some embodiments, the channels are in the form of elliptical transverse electric and magnetic (TEM) transmission lines.

In some embodiments, the channels of the present disclosure have diameters that become wider or narrower across the transmission dielectric probes of the present disclosure. For instance, in some embodiments, the channels of the present disclosure transition from a first shape (e.g., narrow circular shapes) into a second shape (e.g., wider elliptical shapes) across the transmission dielectric probes of the present disclosure.

The channels of the present disclosure can have various components. For instance, in some embodiments, the channels of the present disclosure can include an inner conductor (e.g., inner conductor 23 shown in FIG. 1A). In some embodiments, the channels of the present disclosure include an outer conductor (e.g., outer conductor 22 shown in FIG. 1A).

In some embodiments, the channels of the present disclosure include an inner conductor and an outer conductor. In some embodiments, the outer conductor is coaxial with the inner conductor. In some embodiments, the outer conductor surrounds the inner conductor. In some embodiments, the outer conductor is spaced apart from the inner conductor by a dielectric material.

In some embodiments, the inner conductor is positioned at the center of the channel. In some embodiments, the inner conductor is not positioned at the center of the channel. For instance, in some embodiments, the inner conductor is slightly off the center of the channel. In some embodiments, each of the inner conductor and outer conductor transition from a first shape (e.g., narrow circular shapes) into a second shape (e.g., wider elliptical shapes) across the transmission dielectric probes of the present disclosure.

In some embodiments, the diameter ratio between the inner conductor and outer conductor of the channels of the present disclosure decreases across the transmission dielectric probes of the present disclosure. In some embodiments, the diameter ratio between the inner conductor and outer conductor of the channels of the present disclosure increases across the transmission dielectric probes of the present disclosure. In some embodiments, the diameter ratio between the inner conductor and outer conductor of the channels of the present disclosure remains the same across the transmission dielectric probes of the present disclosure. In some embodiments, the narrowing shapes of the channels of the present disclosure confine a field to a narrower plane for the purposes of focusing an interrogation zone.

The transmission dielectric probes of the present disclosure can include a number of channels. For instance, in some embodiments, the transmission dielectric probes of the present disclosure include one channel. In some embodiments, the transmission dielectric probes of the present disclosure include two channels.

In some embodiments, the transmission dielectric probes of the present disclosure include at least two channels. In some embodiments, the at least two channels are adjacent to one another. In some embodiments, the at least two channels are coaxial to one another.

In some embodiments, the at least two channels of the transmission dielectric probes of the present disclosure have the same channel shape. In some embodiments, the same channel shape is defined by at least one of the same first opening diameter, the same second opening diameter, the same channel geometry across the transmission dielectric probe, or combinations thereof.

In some embodiments, the at least two channels of the transmission dielectric probes of the present disclosure have different channel shapes. In some embodiments, the different channel shapes are defined by at least one of different first opening diameters, different second opening diameters, different channel geometries across the transmission dielectric probe, or combinations thereof.

Channel Openings

The channels of the transmission dielectric probes of the present disclosure can include numerous types of first openings on a first side of the transmission dielectric probe, and numerous types of second openings on a second side of the transmission dielectric probe. In some embodiments, the first opening and the second opening are on opposite ends of the transmission dielectric probe, and the second opening is associated with an outer surface of the transmission dielectric probe.

In some embodiments, the first opening and the second opening have different diameters, different geometries, or combinations thereof. For instance, in some embodiments, the first opening and the second opening have different diameters. In some embodiments, the diameter of the first opening is smaller than the diameter of the second opening. In some embodiments, the diameter of the channel becomes gradually wider as it extends from the first opening to the second opening.

In some embodiments, the diameter of the first opening is larger than the diameter of the second opening. In some embodiments, the diameter of the channel becomes gradually narrower as it extends from the first opening to the second opening.

In some embodiments, the first opening and the second opening have different geometries. For instance, in some embodiments, the first opening is in the form of a circle, and the second opening is in the form of an ellipse. In some embodiments, the first opening is in the form of an ellipse and the second opening is in the form of a circle.

In some embodiments, the first opening and the second opening have different diameters and geometries. For instance, in some embodiments, the first opening has a circular shape and the second opening has an elliptical shape with a larger diameter than the diameter of the first opening.

Additional shapes can also be envisioned for the channels, openings, inner conductors, and outer conductors of the present disclosure. For instance, in some embodiments, the shapes can include, without limitation, a four-leaf clover, a rectangle, a square, or combinations thereof.

Outer Surfaces

In general, the second openings of the channels of the present disclosure extend to an outer surface of the transmission dielectric probes of the present disclosure. The transmission dielectric probes of the present disclosure can include numerous types of outer surfaces. For instance, in some embodiments, the outer surface is flat (e.g., outer surface 21 shown in FIG. 1A). In some embodiments, the outer surface is curved.

In some embodiments, the outer surface serves as an interface between the transmission dielectric probe and a surface of an object (e.g., a tissue or a liquid). In some embodiments, the interface includes an open-circuit interface. In some embodiments, the open-circuit interface reflects most of a transmitted signal back to a generator (e.g., more than 50%, 60%, 70%, 80%, 90%, or 95%).

Analyzers

In some embodiments, the transmission dielectric probes of the present disclosure are also associated with one or more analyzers. As such, in some embodiments, the methods of the present disclosure also include a step of connecting the transmission dielectric probes of the present disclosure to an analyzer. In some embodiments, the methods of the present disclosure also include a step of utilizing the analyzer to display transmitted signals that are received back for evaluation.

In some embodiments, the one or more analyzers include vector network analyzers (VNAs). In some embodiments, the one or more analyzers are associated with the transmission dielectric probes of the present disclosure through one or more cables (e.g., coaxial cables). In some embodiments, the one or more analyzers are associated with the transmission dielectric probes of the present disclosure through a direct connection without any cables.

In some embodiments, the analyzers of the present disclosure (e.g., VNAs) are handheld analyzers (e.g., handheld VNAs) with operating frequencies of up to 3 GHz. In some embodiments, the analyzers (e.g., VNAs) plug directly into a display (e.g., a cell phone, tablet or computer) to display results. For instance, in some embodiments, the analyzers (e.g., VNAs) have USB connections that can be run to either a cell phone, tablet (e.g., iPad) or computer (e.g., a laptop).

In some embodiments, the analyzers of the present disclosure (e.g., VNAs) can display results in real-time. In some embodiments, results related to both amplitude and phase measurements can be displayed on a screen for evaluation, such as by a clinician. In some embodiments, the results (e.g., measurement data) can also be stored. In some embodiments, the results can be applied to signal processing and machine learning computations to help distinguish the physiological state of the different tissues at different layers within the body.

In specific embodiments illustrated in FIG. 1A, transmission dielectric probe 10 is connected to an analyzer through openings 19 and 21. In some embodiments, the connection could be through short cables (e.g., coaxial cables) that run between openings 19 and 21 and the connectors of an analyzer. In some embodiments, the connection could be direct without the utilization of any cables.

Placement of Transmission Dielectric Probes on Surfaces of Objects

The outer surfaces of the transmission dielectric probes of the present disclosure may be placed on surfaces of various objects. For instance, in some embodiments, the object is a tissue. In some embodiments, the object is a soft tissue. In some embodiments, the soft tissue includes, without limitation, muscles, tendons, ligaments, fascia, nerves, fibrous tissues, breast tissue, thyroid tissue, fat, blood vessels, synovial membranes, and combinations thereof.

In some embodiments, the object is a hard object. In some embodiments, the hard object is a hard tissue. In some embodiments, the hard tissue includes, without limitation, bone, tooth enamel, dentin, cementum, and combinations thereof.

In some embodiments, the tissue is derived from a subject. In some embodiments, the tissue is part of a subject.

In some embodiments, the object is a liquid. In some embodiments, the liquid is a viscous liquid. In some embodiments, the liquid is derived from a tissue, such as a soft tissue. In some embodiments, the liquid is part of a tissue.

In some embodiments, pressure may be applied to a surface of an object during the placement of the transmission dielectric probes of the present disclosure on the surface of the object. In some embodiments, pressure may be applied to an interface between a surface of an object and a transmission dielectric probe in order to eliminate air gaps and thereby allow a deeper evaluation of the object. For instance, in some embodiments, the transmission dielectric probes of the present disclosure may be pushed onto a surface of an object.

In some embodiments, gels may be applied to a surface of an object during the placement of the transmission dielectric probes of the present disclosure on the surface of the object. In some embodiments, gels may be applied to an interface between a surface of an object and a transmission dielectric probe in order to eliminate any air gaps and thereby allow a deeper evaluation of the object.

Transmission of Signals

The transmission dielectric probes of the present disclosure may be utilized to transmit various types of signals through surfaces of objects. For instance, in some embodiments, the transmitted signal includes an electromagnetic signal. In some embodiments, the electromagnetic signal includes a microwave field.

Receiving Transmission Signals Back

The transmission dielectric probes of the present disclosure may be utilized to receive transmission signals back in various manners. For instance, in some embodiments, transmission signals are transmitted from a first channel of a transmission dielectric probe onto a surface and into an object and then received back through a second channel of the transmission dielectric probe. In some embodiments, the first channel receives reflected transmission signals, such as transmission signals that are reflected back from a surface.

In more specific embodiments, transmission signals travel through a first channel and an open circuit interface between a surface of an object and the outer surface of the transmission dielectric probe. Thereafter, the open circuit interface reflects a majority of the transmission signal (e.g., 99%) back to a generator. However, a minority of the transmission signals (e.g., 1%) travel through the surface, into the object, and back to the dielectric transmission probes of the present disclosure through second channels.

Transmission signals can be received back by the transmission dielectric probes of the present disclosure in various forms. For instance, in some embodiments, the transmission signals of the present disclosure are received back as a function of phase v. frequency, amplitude v. frequency, magnitude v. frequency, or combinations thereof. In some embodiments, the transmission signals of the present disclosure are received back as a function of phase v. frequency. In some embodiments, the transmission signals of the present disclosure are received back as a function of amplitude v. frequency. In some embodiments, the transmission signals of the present disclosure are received back as a function of amplitude v. frequency and phase v. frequency.

Evaluation of Properties of Objects

Transmitted signals that are received back by the transmission dielectric probes of the present disclosure can be utilized to evaluate various properties of an object in various manners. For instance, in some embodiments, the evaluation includes a calculation of a property of the object (e.g., dielectric properties of the object). In some embodiments, the evaluation includes the characterization of the object directly from the transmitted signals.

In some embodiments, the evaluation occurs at a single time point (e.g., a single evaluation at a given time). In some embodiments, the evaluation occurs at multiple time points (e.g., two or more different time points over a period of time).

In some embodiments, the methods of the present disclosure evaluate the property of an object by comparing the transmitted signals received back from the object with data that are associated with known properties. In some embodiments, the methods of the present disclosure evaluate the property of an object by utilizing an algorithm to evaluate the transmitted signals received back from the object.

In some embodiments, the algorithm is a machine learning algorithm. In some embodiments, the machine learning algorithm is utilized to analyze a broad spectrum of transmitted signals received back from the object in order to evaluate the property of the object. In some embodiments, the machine learning algorithm is utilized to analyze a broad spectrum of transmitted signals received back from the object at a single time point (e.g., at a set time) in order to evaluate the property of the object. In some embodiments, the machine learning algorithm is utilized to analyze a broad spectrum of transmitted signals received back from the object at multiple time points (e.g., over a period of time, such as two or more different time points) in order to evaluate the property of the object. In some embodiments where the object is a tissue, the transmitted signals can be utilized to evaluate a property of the tissue. In some embodiments, the evaluated property of the tissue includes, without limitation, water content, fat content, tumor growth, malignancy, tissue damage, tissue dielectric properties, or combinations thereof.

In some embodiments, the evaluated property of the tissue can be utilized to differentiate between different classes of tissues, such as fat and muscle tissues. In some embodiments, the evaluated property of a tissue can be utilized to differentiate between different layers of tissues. In some embodiments, the evaluated property of a tissue can be utilized to characterize tissue types at different layers within the tissue (e.g., both the thicknesses and characteristics of the tissue, such as skin, subcutaneous fat, muscle, and/or bone layers).

In some embodiments, the evaluated property of a tissue can be utilized to detect changes in a property of a tissue over time. For instance, in some embodiments, the evaluated property of a tissue can be utilized to detect a change in bone density over time, a change in fat content of muscle over time, a change in water content of muscle over time, or combinations thereof.

In some embodiments, the evaluated property of a tissue can be utilized to detect a condition associated with a tissue. In some embodiments, the condition includes, without limitation, cancer, breast cancer, edema, stroke, osteoporosis, sarcopenia, or combinations thereof.

In some embodiments, the evaluated property of a tissue can be utilized to make a treatment decision. In some embodiments, the treatment decision is the treatment of a disease or condition in the subject. In some embodiments, the subject is a human being suffering from the disease or condition.

In some embodiments, machine learning algorithms may be utilized to evaluate a property of a tissue. For instance, in some embodiments, machine learning algorithms may be utilized to analyze a broad spectrum of transmitted signals received back from an object at a single time point or at multiple time points (e.g., over a period of time) in order to evaluate the property of the tissue.

In some embodiments, the methods and transmission dielectric probes of the present disclosure are utilized to determine tissue health. In some embodiments, the tissue health is determined by distinguishing healthy tissue from compromised tissue. In some embodiments, the transmission properties of the transmission dielectric probes of the present disclosure could be especially specific in distinguishing healthy tissue from diseased or otherwise compromised tissue.

In some embodiments, the methods and transmission dielectric probes of the present disclosure can be utilized to detect cancer, such as breast cancer. In some embodiments, the methods and transmission dielectric probes of the present disclosure detect cancer by distinguishing between malignant and benign lumps, such as malignant and benign breast lumps. In particular, tissues containing breast cancer have high dielectric properties when compared to that of normal tissue.

In some embodiments, the methods and transmission dielectric probes of the present disclosure can be utilized to detect edema. In particular, edema under the skin has high water content which would have very different properties than that for normal skin and the subcutaneous fat.

In some embodiments, the methods and transmission dielectric probes of the present disclosure can be utilized in imaging for detection of stroke. In particular, blood has very different properties than that for normal brain tissue, which could be utilized for stroke detection in accordance with the methods of the present disclosure.

In some embodiments, the methods and transmission dielectric probes of the present disclosure can be utilized to detect osteoporosis. For instance, in some embodiments, the transmission dielectric probes of the present disclosure can be utilized to distinguish between normal and osteoporotic bone. In particular, normal and osteoporotic bone have different properties that could be distinguished by the methods and transmission dielectric probes of the present disclosure.

In some embodiments, the transmission dielectric probes of the present disclosure can be utilized in rehabilitation medicine. In some embodiments, utilization in rehabilitation medicine may involve the utilization of the transmission dielectric probes of the present disclosure in assessing bone and muscular health in subjects (e.g., elderly patients) after surgical procedures, which may in turn provide useful therapeutic information. In some embodiments, utilization in rehabilitation medicine may involve the utilization of the transmission dielectric probes of the present disclosure for diagnosis during surgery.

For instance, the dielectric properties for muscle and bone change during different stages of rehabilitation. In particular, muscles under atrophy have higher fat infiltration and lower dielectric properties than rehabilitated muscles. The methods and transmission dielectric probes of the present disclosure can be utilized to detect such differences in order to monitor the progress of rehabilitation.

Similarly, bone density can diminish considerably after an injury. Moreover, bone dielectric properties change as a function of density. As a result, the methods and transmission dielectric probes of the present disclosure can be utilized to detect changes in bone density in order to monitor the progress of rehabilitation.

In some embodiments, the methods and transmission dielectric probes of the present disclosure can be utilized to detect sarcopenia. Sarcopenia is characterized by infiltration of large amounts of fat into muscles. On the other hand, normal muscle has a high amount of water. Water has very high dielectric properties, while fat has very low dielectric properties. Accordingly, magnitude and phase signatures for signals transmitting through normal and sarcopenic muscle are expected to be quite different. Likewise, low dielectric properties mean the tissue is closer to fat while high properties mean it is closer to muscle. In the case of sarcopenia, lower properties means it is closer to being dominated by fat (i.e. quite sarcopenic), while higher properties would be closer to normal muscle. These differences will enable the methods and transmission dielectric probes of the present disclosure to detect sarcopenia.

The methods and transmission dielectric probes of the present disclosure can also have numerous additional applications. For instance, in some embodiments where an object is a liquid, the methods and transmission dielectric probes of the present disclosure can be utilized to evaluate a property of the liquid. In some embodiments, the property of the liquid includes, without limitation, the type of liquid, the concentration of the liquid, the dielectric properties of the liquid, or combinations thereof. In some embodiments, machine learning algorithms may be utilized to evaluate a property of the liquid.

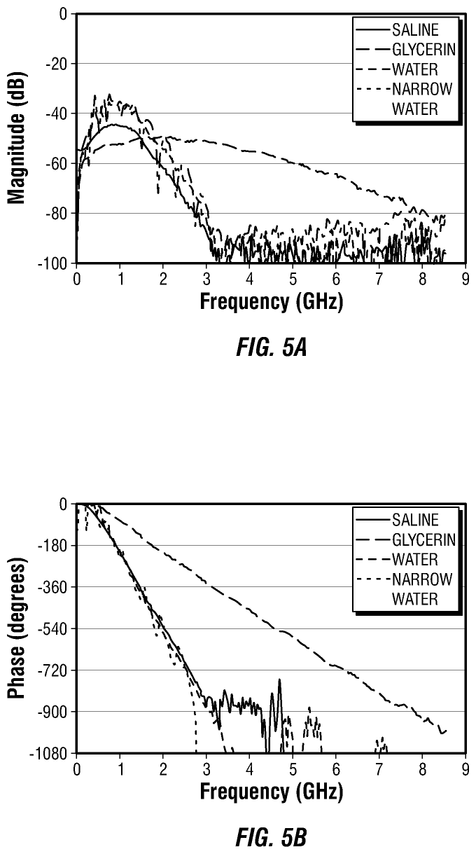

For instance, as illustrated in FIGS. 5A and 5B, different liquids (e.g., water and glycerin) have different dielectric properties with unique magnitude and phase signatures at different concentrations. Such differences in dielectric properties can be utilized to identify a liquid and determine the concentration of the liquid in accordance with the methods of the present disclosure.

Fabrication of Transmission Dielectric Probes

Additional embodiments of the present disclosure pertain to methods of making the transmission dielectric probes of the present disclosure. In some embodiments, the transmission dielectric probes of the present disclosure are fabricated through the utilization of three-dimensional (3D) printing. In some embodiments, a metal printer may be utilized to print the transmission dielectric probes of the present disclosure.

Properties and Advantages

The transmission dielectric probes of the present disclosure can have numerous advantageous properties. For instance, in some embodiments, movement of cables associated with the transmission dielectric probes of the present disclosure has virtually no effect on the performance of the transmission dielectric probes.

In some embodiments, the transmission dielectric probes of the present disclosure have surface penetration depths of at least one centimeter. As such, in some embodiments, the transmission dielectric probes of the present disclosure are capable of transmitting signals that can travel through a thin layer of skin and through a subcutaneous layer of fat.

In some embodiments, the transmission dielectric probes of the present disclosure have broad bandwidths. For instance, in some embodiments, the transmission dielectric probes of the present disclosure have bandwidths of more than about 5%. In some embodiments, the transmission dielectric probes of the present disclosure have bandwidths of more than about 10%. In some embodiments, the transmission dielectric probes of the present disclosure have bandwidths of more than about 25%. In some embodiments, the transmission dielectric probes of the present disclosure have bandwidths of more than about 50%. In some embodiments, the transmission dielectric probes of the present disclosure have bandwidths of more than about 75%. In some embodiments, the transmission dielectric probes of the present disclosure have bandwidths of more than about 100%. In some embodiments, the transmission dielectric probes of the present disclosure have bandwidths of more than about 150%. In some embodiments, the transmission dielectric probes of the present disclosure have bandwidths of more than about 175%. In some embodiments, the transmission dielectric probes of the present disclosure have bandwidths of more than about 190%.

In some embodiments, the bandwidths of the transmission dielectric probes of the present disclosure are defined as the frequency range for which the measured magnitude is above a noise floor. In some embodiments, the transmission dielectric probes of the present disclosure have bandwidths ranging from about 1 MHz to about 50 GHz. In some embodiments, the transmission dielectric probes of the present disclosure have bandwidths ranging from about 100 MHz to about 5 GHz. In some embodiments, the transmission dielectric probes of the present disclosure have bandwidths of more than about 50 MHz. In some embodiments, the transmission dielectric probes of the present disclosure have bandwidths of more than about 100 MHz. In some embodiments, the transmission dielectric probes of the present disclosure have bandwidths of more than about 500 MHz. In some embodiments, the transmission dielectric probes of the present disclosure have bandwidths of more than about 1 GHz. In some embodiments, the transmission dielectric probes of the present disclosure have bandwidths of more than about 2 GHz. In some embodiments, the transmission dielectric probes of the present disclosure have bandwidths of more than about 3 GHz. In some embodiments, the transmission dielectric probes of the present disclosure have bandwidths of more than about 5 GHz.

In some embodiments, the broad bandwidths associated with the transmission dielectric probes of the present disclosure provide the added advantage of extensive spectral content. In some embodiments, the extensive spectral content can further be used in diagnosis and distinguishing features at different depths.

For instance, in some embodiments, signals for the different spans of a broad bandwidth will have different behaviors. In some embodiments, signals for the lower frequency span will penetrate deeper into a tissue because the attenuation is less than that for the higher frequency span. In some embodiments, such a differential characteristic could allow for a more in depth understanding of what the thicknesses of the different tissue strata are and what the dielectric properties of each tissue strata are, especially when machine learning algorithms are utilized.

In some embodiments, the transmission dielectric probes of the present disclosure are operable over a broad range of frequencies. For instance, in some embodiments, the transmission dielectric probes of the present disclosure are operable at frequencies that range from about 1 MHz to about 10 GHz. In some embodiments, the transmission dielectric probes of the present disclosure are operable at frequencies that range from about 100 MHz to about 10 GHz.

In some embodiments, the channels of the transmission dielectric probes of the present disclosure have different impedance values at different ends. For instance, in some embodiments, the impedance value of a channel at the point where it interfaces with a surface (e.g., a tissue) is higher (e.g., 300-400 ohms) than the impedance value of the channel at the point where it connects with a connector end (e.g., 50-75 ohms). In some embodiments, such properties are advantageous because they enable transmission of a signal into a tissue while minimizing reflection at a surface.

In some embodiments, impedance values of the transmission dielectric probes of the present disclosure are calculated by the following formula:

$${Z}_{o} = \frac{138 \times {log}_{10} ( \frac{D}{d} )}{\sqrt{{ε}_{r}}}$$

In the aforementioned formula, Zₒ is the characteristic impedance, D is the outer conductor diameter, d is the inner conductor diameter and εᵣ is the relative permittivity of the dielectric material separating the two conductors.

For instance, in a microwave realm where one can measure signals down to especially low power levels, there is still sufficient power fringing out beyond an open circuit to be coupled to an adjacent receiving open-circuit. In traversing from the one channel opening to the next, the signal propagates through a substantial amount of a surface (e.g., tissue) to the point that it can be used for interrogation. This type of transmission is considered non-standard in the microwave arena because the return-loss or reflected signal is large, implying that the transmission signal would be very low.

In some embodiments, the transmission dielectric probes of the present disclosure are compact in shape. In some embodiments, the transmission dielectric probes of the present disclosure are handheld.

In some embodiments, the transmission dielectric probes of the present disclosure advantageously build on technology in three prior inventions: (1) an oversized coaxial reflection probe (for testing wine) (U.S. Pat. No. 10,113,979); (2) an oversized coaxial, side-by-side transmission probe (for testing wine) (PCT/US2019/044120); and (3) an opposing coaxial transmission probe (for measuring bone strength of vertebrae at point of care) (PCT/EP2019/063228). In some embodiments, the advantages of the transmission dielectric probes of the present disclosure include, without limitation: (1) further and deeper signal penetration into a surface (e.g., a tissue); (2) less susceptibility to cable motion artifacts; (3) reduction of the effects of multi-path signal corruption; and (4) more amenability for clinical applications.

As described previously, a more specific advantage of the transmission dielectric probes of the present disclosure is that they receive back transmission signals instead of reflected signals. This provides substantial advantages in that the surface penetration depth is greater and the transmission signals are not prone to artifacts because of simple feed cable motion-the latter of which would make it impossible to use for any clinical application. In fact, these limitations have single-handedly restricted the use of transmission dielectric probes in clinical applications, even when the specific nature of the tissue dielectric properties have been well-known for decades.

### Additional Embodiments

Reference will now be made to more specific embodiments of the present disclosure and experimental results that provide support for such embodiments. However, Applicants note that the disclosure below is for illustrative purposes only and is not intended to limit the scope of the claimed subject matter in any way.

### EXAMPLE 1

### Development and Use of Transmission Dielectric Probes

In this Example, Applicants have applied advanced computer-aided design and three-dimensional printing techniques to fabricate two initial prototypes which show considerable promise in early tests. Applicants' strategy was to form two adjacent elliptical TEM mode interfaces to attempt to confine the fields to a narrower plane and focus the interrogation zone. To achieve this, Applicants used three-dimensional printing techniques.

Instead of the taper being from small diameter circles (near the connector) to large diameter circles (near the bottle) (as was the case for prior inventions), Applicants devised a taper from the small circles to large ellipses. Computer design tools such as SolidWorks are fully capable of making such designs. Moreover, the resulting models can be directly fabricated in three-dimensional printing.

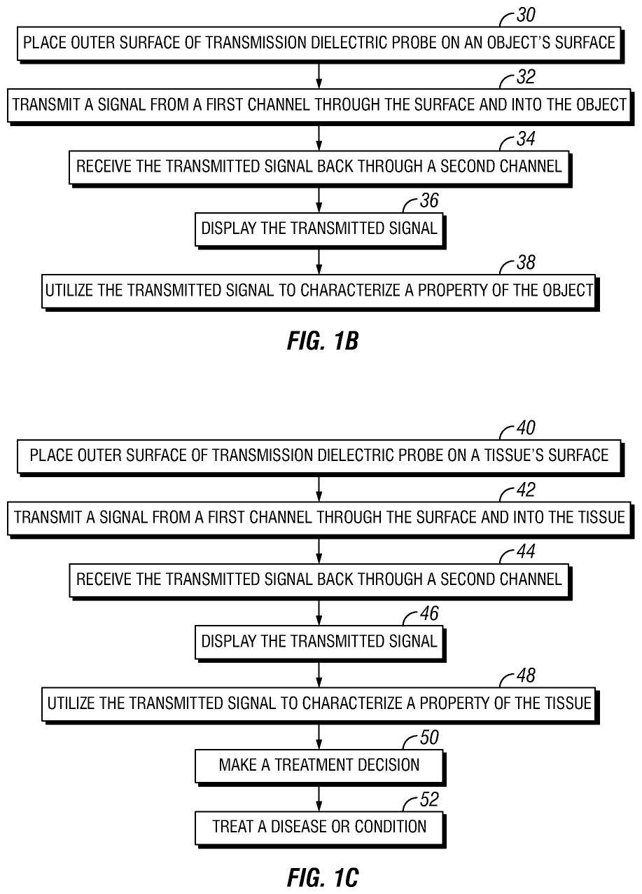

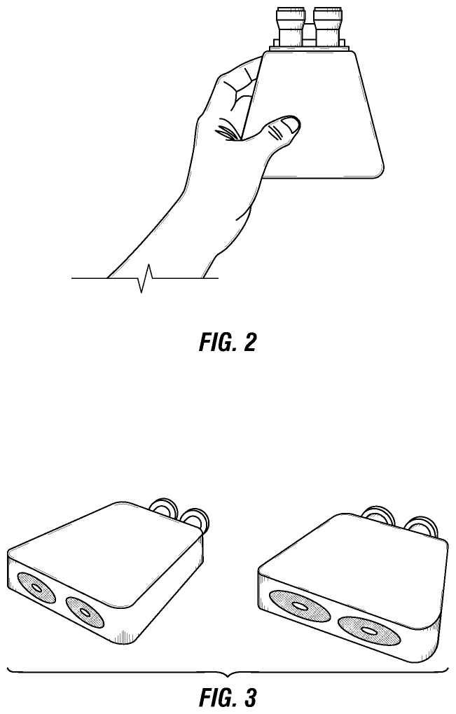

Applicants printed the outer housing and the tapered inner conductors in metal, making allowances for a temporary structure to hold the inner conductor in place. Applicants then used a slow curing epoxy as the insulator between the center and outer conductors. Once the epoxy cured, Applicants machined off the temporary structures and added the coaxial connectors. The designs are illustrated in FIGS. 2-3.

Applicants focused an interrogation zone by first defining a probing plane as the plane passing through the centers of the inner conductors of both openings and normal to the aperture surfaces. This probing plane can be considered to have a thickness which is roughly defined as the height of each aperture. By narrowing these heights, the thickness of the imaging plane gets thinner. A thinner probing plane could be useful to a clinician with respect to possibly identifying the location of an abnormality and assessing the physiological state of a specific tissue zone.

Based on the test results, Applicants confirmed that the movement of the cables has virtually no effect on the performance. Testing with just homogeneous liquids has shown that Applicants can easily distinguish between classes of liquids. Pilot studies on sample human tissue suggests that Applicants can readily distinguish between measurements near fat, muscle and bone.

The use of both the spectral phase and magnitude measurements appears to be quite unique for different types. Accordingly, Applicants envision that, in a dynamic/diagnostic setting (i.e., where Applicants can manually scan the probe over the skin surface from known normal tissue to a suspected malady), Applicants will be able to provide useful clinical data.

### Example 2

### Use of Transmission Dielectric Probes in Repeatability Measurements

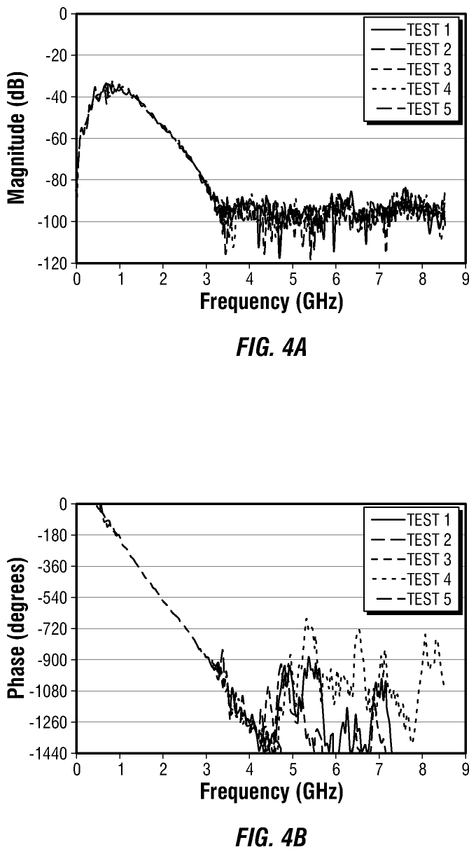

In this Example, Applicants demonstrated that measurements with the transmission dielectric probes described in Example 1 were repeatable when they were dipped in a bath of saline. Applicants used a standard Agilent vector network analyzer (VNA) and the settings for its default calibration. Two 4 foot cables were connected from the two ports on the VNA to the two connectors on the probe. The probes were subsequently partially submerged (just covering the flat surface of the probe) into a bath of saline and an S21 measurement was taken (i.e., a transmission measurement indicating that the signal was transmitted from port 1 on the VNA and received at port 2.) This process was repeated 5 times and the magnitude and phase results are plotted in FIGS. 4A and 4B, respectively.

Applicants note that the saline is very lossy so that the high frequency measurements go into the noise. However, up to about 3 GHz, the measurements are clear and repeatable. Moreover, the phase data becomes insignificant once the amplitude dips below the noise floor—in this case around 3 GHz.

### Example 3

### Use of Transmission Dielectric Probes for Measurements in Different Liquids

In this Example, Applicants aimed to confirm that the transmission dielectric probes described in Example 1 provided distinct measurements for different dielectric liquids. For this Example, Applicants compared water, saline and glycerin. The results, which represent S21 measurements with the same VNA and the same default calibration settings in Example 2, are shown in FIGS. 5A and 5B in terms of amplitude and phase measurements, respectively.

It should be noted that there are two sets of measurements for water. In one case, Applicants used a large bath (roughly a 1.5′ square by about 1′ high). The second was for using a narrow Plexi-glas cylindrical tube (roughly 6″ diameter).

The phase (FIG. 5B) and magnitude (FIG. 5A) measurements for each are fairly similar except that those for the narrow water measurements have considerable ripples in the curves. These ripples are due to standing waves whereby the signals reflect back and forth between the probe and tube walls.

The phase curves (FIG. 5B) for all of the water and saline baths are very similar and the associated curve for glycerin is quite different (i.e., a less steep slope). Such results effectively differentiate high permittivity liquids (water and saline) from low permittivity liquids (glycerin).

For the magnitude curves (FIG. 5A), each curve has a general up-side-down parabola. Those for the water-based liquids have a distinct peak at a lower frequency and the signal strength effectively goes to the noise floor (in this case about −90 dB) by about 3 GHz. The glycerin curve is a much broader parabola with a higher frequency peak. It is noteworthy that the parabola for the saline is very similar to that for water except that it is lower. In this case, the only difference between water and saline is the salt content. The added salt dramatically increases the conductivity of the liquid and subsequently increases the attenuation. This fully accounts for why the saline curve is lower than that for water.

### Example 4

### Use of Transmission Dielectric Probes for Measurements at Different Penetration Depths

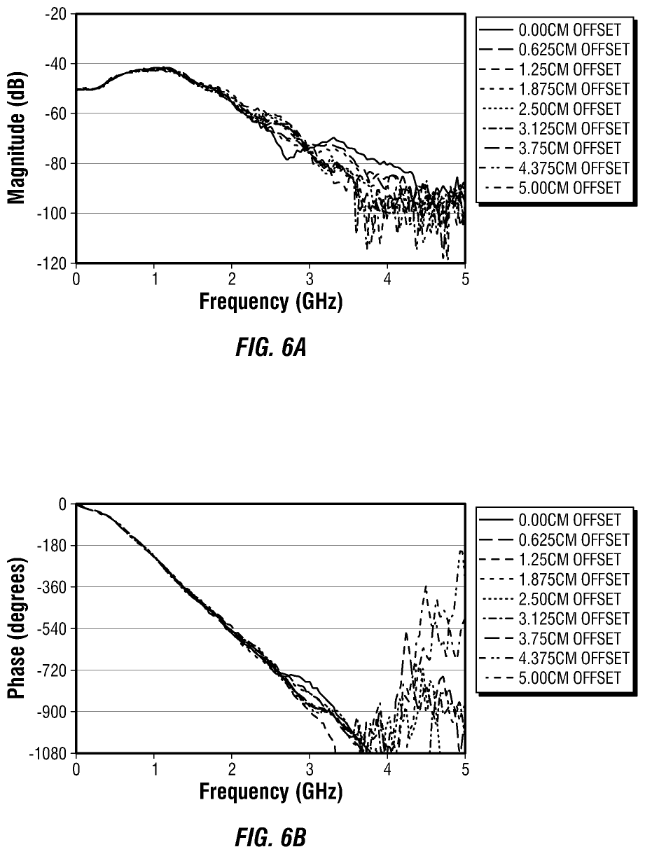

In this Example, Applicants aimed to confirm that the transmission dielectric probes described in Example 1 can see features at deeper depths than conventional reflection-based probes. To test this hypothesis, Applicants built a fixture so that the transmission dielectric probe could be suspended in a liquid bath, but also moved along horizontally. Next, Applicants placed a vertical ridge of Plexiglas in the middle of the tank (very different properties from that of the bath—Applicants would expect its presence to disrupt the measurements). Thereafter, Applicants tested for what heights the presence of the Plexiglas impacted the measurements. The results are shown in FIGS. 6A-B.

The graphs in FIGS. 6A-B show the magnitude and phase measurements for the case where the probes are 2.3 cm above the ridge. The different curves are for the different horizontal offsets from the ridge—i.e. further offsets from the ridge should show no impact on the measurements. Notably, the measurements above about 3.5 GHz are essentially noise. For all of the measurements with offsets of 2.5 cm or greater, the curves overlap. However, for the closer distances, there are perturbations in both magnitude and phase at the higher frequencies, indicating that the Plexiglas is impacting the measurements.

The aforementioned results indicate that the probes of the present disclosure can conduct measurements at least 2.3 cm into a liquid. Applicants note that, due to their lossy nature, the liquids in this Example are essentially a worst case scenario that Applicants would encounter in tissue. However, for the body, the signals first encounter a lossy skin layer, but right underneath is a thicker low loss, fat layer. Applicants actually expect the probes of the present disclosure to see even deeper into real tissue.

### Example 5

### Use of Transmission Dielectric Probes for Actual Body Measurements

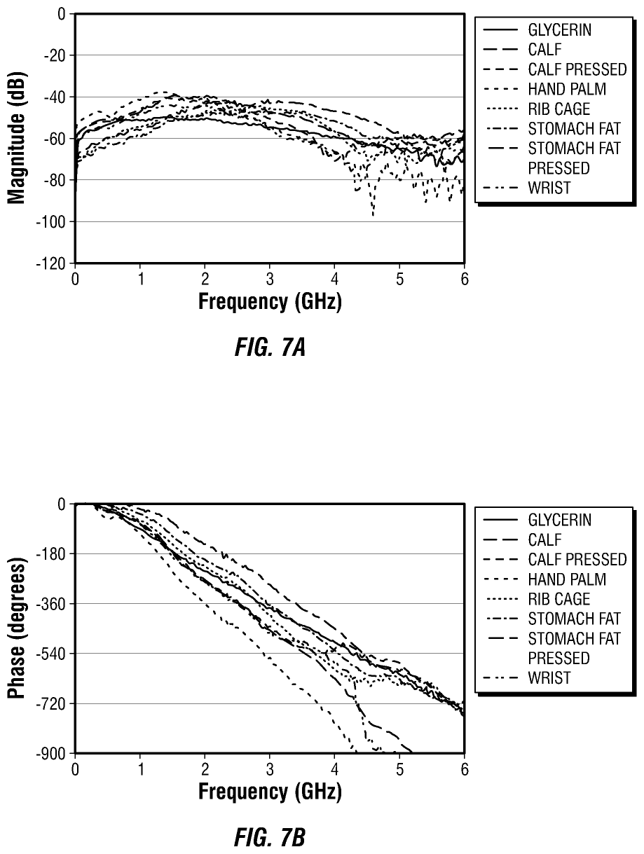

In this Example, Applicants used the transmission dielectric probes of Example 1 to make various measurements on the body. The results are shown in FIGS. 7A-7B.

Notably, there is also a curve for a glycerin bath to be used as a low permittivity reference. Moreover, Applicants were able to obtain viable measurements (i.e., above the noise floor) up to higher frequencies than Applicants saw for the earlier saline measurements.

The aforementioned results affirm Applicants' hypothesis that signals from the transmission dielectric probes of the present disclosure travel deeper because the subcutaneous fat layer has lower loss. Applicants grouped the aforementioned measurements into two categories: (a) fattier—i.e., lower permittivity and lower conductivity; and (b) higher water content—i.e., higher permittivity and higher conductivity. The two stomach fat cases appear the closest to the glycerin curves than the others.

Applicants note that, for the amplitudes, each curve starts at a low level near zero frequency, gradually reaching a peak at a higher frequency and then finally decreasing again at higher frequencies. Where each curve hits its peak is significant. The peaks for the glycerin and fat are at higher frequencies, while many of the others are at lower frequencies.

Moreover, the curve representing the wrist is distinguishable because it has less subcutaneous fat with lots of muscle and tendons. Therefore, the wrist would resemble the higher water content tissues. The wrist, calf and hand palm measurements (the part of the palm Applicants measured would also have had low subcutaneous fat) generally have amplitude peaks at a much lower frequency than for the fattier tissue. The phase measurements also appear to bear out these distinctions.

### Example 6

### Use of Transmission Dielectric Probes to Characterize Different Sites of a Body

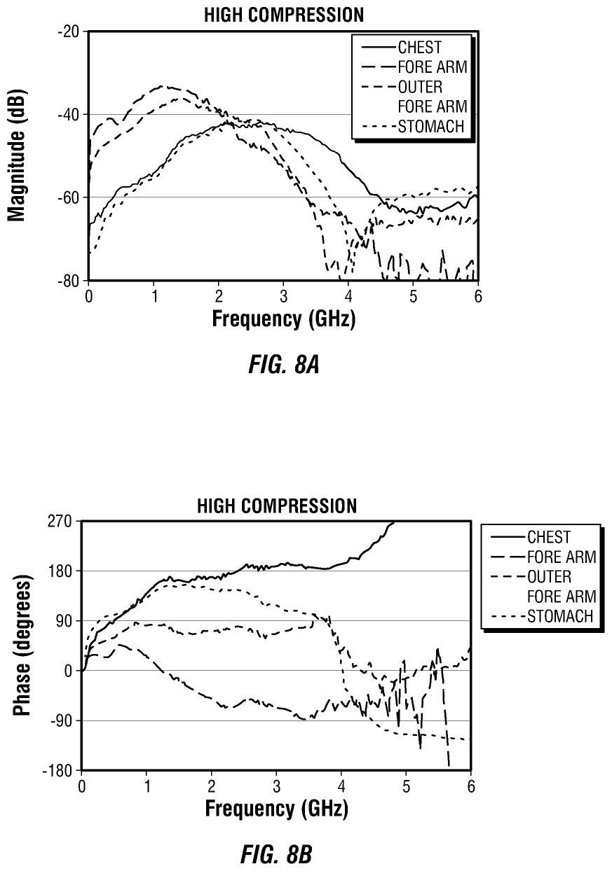

FIGS. 8A and 8B show some sample magnitude and phase measurements for different sites on the body. In this case, Applicants implemented a calibration so that the phase slopes were not so extreme (i.e., it is easier to differentiate the phase plots when the steep slope is removed).

An important feature in this Example is the difference between the slopes of each. In this Example, there are two sites on the body that Applicants would consider fattier and two more muscular (i.e., higher water content). The fattier ones are the stomach and chest. The more muscular ones are the forearm and upper forearm.

For the magnitude plots (FIG. 8A), the more muscular sites of the body have a lower frequency peak—the parabola is shifted to the left. For the fattier sites of the body, the parabolas are shifted to the right. The differences between the two sets is quite noticeable. For the phase plots (FIG. 8B), the ones for the more muscular ones are lower while the fattier ones are higher. It appears that the probe can readily differentiate between these two classes of tissues. The spectral curves for each are quite unique. Long term, these could provide information so that Applicants can extract information directly related to the underlying tissue and minimize the effects of the skin and fat on the measurements.

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

What is claimed is:

1. A transmission dielectric probe comprising:
at least two adjacent channels extending across the transmission dielectric probe, wherein each of the at least two adjacent channels comprises:
a first opening on a first side of the transmission dielectric probe, and
a second opening on a second side of the transmission dielectric probe,
wherein the first opening and the second opening are on opposite ends of the transmission dielectric probe,
wherein the second opening is associated with an outer surface of the transmission dielectric probe, and
wherein the first opening and the second opening have different diameters, different geometries, or combinations thereof.

2. The transmission dielectric probe of claim 1, wherein the first opening and the second opening have different diameters, wherein the diameter of the first opening is smaller than the diameter of the second opening, and wherein the diameter of the channel becomes gradually wider as it extends from the first opening to the second opening.

3. (canceled)

4. The transmission dielectric probe of claim 1, wherein the first opening and the second opening have different geometries, wherein the first opening is in the form of a circle, and wherein the second opening is in the form of an ellipse.

5. (canceled)

6. The transmission dielectric probe of claim 1, wherein each of the at least two adjacent channels is enclosed within the transmission dielectric probe, wherein each of the at least two adjacent channels comprises an inner conductor and an outer conductor, wherein the outer conductor is coaxial with the inner conductor.

7. (canceled)

8. The transmission dielectric probe of claim 6, wherein the inner conductor is not positioned at the center of the channel.

9. The transmission dielectric probe of claim 1, wherein the at least two adjacent channels have the same channel shape, wherein the same channel shape is defined by at least one of the same first opening diameter, the same second opening diameter, the same channel geometry across the transmission dielectric probe, or combinations thereof, and wherein the outer surface serves as an interface between the transmission dielectric probe and a surface of an object.

10. (canceled)

11. The transmission dielectric probe of claim 1, further comprising one or more analyzers associated with the transmission dielectric probe, wherein the one or more analyzers is associated with the transmission dielectric probe through one or more cables.

12. (canceled)

13. The transmission dielectric probe of claim 1, wherein the transmission dielectric probe has a surface penetration depth of at least one centimeter.

14. A method of operating a transmission dielectric probe, said method comprising:
placing the transmission dielectric probe on a surface of an object, wherein the transmission dielectric probe comprises:
at least two channels, wherein each of the at least two channels extends across the transmission dielectric probe, and wherein each of the at least two channels comprises:
a first opening on a first side of the transmission dielectric probe, and
a second opening on a second side of the transmission dielectric probe,
wherein the first opening and the second opening are on opposite ends of the transmission dielectric probe,
wherein the second opening is associated with an outer surface of the transmission dielectric probe, and
wherein the outer surface is placed on the surface of the object;
transmitting a signal from a first channel of the at least two channels through the surface and into the object; and
receiving the transmitted signal back through a second channel of the at least two channels.

15. The method of claim 14, further comprising a step of connecting the transmission dielectric probe to an analyzer and utilizing the analyzer to display the transmitted signal received back from the second channel, wherein the transmitted signal is received back as a function of phase v. frequency, amplitude v. frequency, magnitude v. frequency, or combinations thereof.

16-17. (canceled)

18. The method of claim 14, wherein the transmitted signal comprises an electromagnetic signal.

19. The method of claim 14, wherein the transmitted signal received back from the second channel is utilized to evaluate a property of the object.

20. The method of claim 14, wherein the object is a tissue of a subject wherein the tissue is selected from the group consisting of hard tissue, soft tissue, or combinations thereof, wherein the transmitted signal received back from the second channel is utilized to evaluate a property of the tissue, wherein the property of the tissue is selected from the group consisting of water content, fat content, tumor growth, malignancy, tissue damage, tissue dielectric properties, or combinations thereof.

21-23. (canceled)

24. The method of claim 20, wherein the evaluated property is utilized to distinguish healthy tissue from compromised tissue, differentiate between different classes of tissues, differentiate between different layers of tissues, detect changes in a property of a tissue over time, detect a condition associated with the tissue, or combinations thereof.

25. The method of claim 20, wherein the evaluated property is utilized to detect a condition associated with the tissue, and wherein the condition is selected from the group consisting of cancer, breast cancer, edema, stroke, osteoporosis, sarcopenia, or combinations thereof.

26. The method of claim 20, wherein the evaluated property is utilized to make a treatment decision, wherein the treatment decision is the treatment of a disease or condition in the subject.

27. (canceled)

28. The method of claim 14, wherein the object is a liquid, wherein the transmitted signal received back from the second channel is utilized to evaluate a property of the liquid, and wherein the property of the liquid is selected from the group consisting of the type of liquid, the concentration of the liquid, the dielectric property of the liquid, or combinations thereof.

29-30. (canceled)

31. The method of claim 14, wherein the first opening and the second opening have different diameters, wherein the diameter of the first opening is smaller than the diameter of the second opening, wherein the diameter of the channel becomes gradually wider as it extends from the first opening to the second opening, wherein the first opening is in the form of a circle, and wherein the second opening is in the form of an ellipse.

32-33. (canceled)

34. The method of claim 14, wherein each of the at least two channels is enclosed within the transmission dielectric probe, wherein each of the at least two channels comprises an inner conductor and an outer conductor, wherein the outer conductor is coaxial with the inner conductor, and wherein the inner conductor is not positioned at the center of the channel.

35-36. (canceled)

37. The method of claim 14, wherein each of the at least two channels have the same channel shape, wherein the same channel shape is defined by at least one of the same first opening diameter, the same second opening diameter, the same channel geometry across the transmission dielectric probe, or combinations thereof, wherein the outer surface serves as an interface between the transmission dielectric probe and the surface of the object, and wherein transmission dielectric probe has a bandwidth of more than about 50%.

38-40. (canceled)
